(19) United States
(12) Patent Application Publication
(10) Pub. No.: US 2020/0268370 A1
(43) Pub. Date: Aug. 27, 2020

(54) CARTILAGE AND SOFT TISSUE FIXING DEVICE

(71) Applicants: Wei Ding, Kunming (CN); Xiaotian TAN, Kunming (CN)

(72) Inventors: Wei DING, KUNMING (CN); Xiaotian TAN, KUNMING (CN); Hongbo TAN, KUNMING (CN)

(73) Assignees: Wei Ding; Xiaotian Tan

(21) Appl. No.: 15/931,518

(22) Filed: May 13, 2020

Related U.S. Application Data
(63) Continuation of application No. PCT/CN2019/087147, filed on May 16, 2019.

(30) Foreign Application Priority Data
May 22, 2018 (CN) 201810496155.7

(51) Int. Cl. A61B 17/04

(52) U.S. Cl. CPC A61B 17/0401; A61B 2017/0464; A61B 2017/0403

(57) ABSTRACT

A cartilage and soft tissue fixing device comprises: a plurality of anchors, the anchors being composed of columns and vertebral apexes, and the columns being evenly provided with bone growth holes and/or bone growth slots, wherein the columns are further evenly provided with anti-falling limiting slots, and the columns are provided with line passing holes or line clamping slots near tail ends; and network bridges, the network bridges comprising peripheral bridges and inscribed bridges, the inscribed bridges being connected in the peripheral bridges and the anchors being connected to the peripheral bridges by means of the line passing holes or the line clamping slots, wherein the network bridges may effectively cover a cartilage or soft tissue area to be fixed, and prevent the cartilage or soft tissue from falling off.

### CROSS-REFERENCE TO RELATED APPLICATIONS

This application is a continuation of International Patent Application No. PCT/CN2019/087147 with a filing date of May. 16, 2019, designating the United States, now pending, and further claims priority to Chinese Patent Application No. 201810496155.7 with a filing date of May 22, 2018. The content of the aforementioned applications, including any intervening amendments thereto, are incorporated herein by reference.

### TECHNICAL FIELD

The present invention belongs to the technical field of medical equipment, and particularly relates to a cartilage and soft tissue fixing device of network bridge-shaped structure. The network bridge-shaped structure can fix the cartilage tissue through a network structure, and can provide the biomechanical strength of a cartilage repair period through a bridge structure.

### BACKGROUD OF THE PRESENT INVENTION

Traditional cartilage or soft tissue repair and fixation surgeries, or the surgeries that use tissue engineering cartilage newly developed in recent years to repair joint or meniscus defects caused by sports injuries or arthritis, and to repair or reconstruct the defect or loss of the ear, nose, trachea and tarsus caused by tumor resection, trauma or congenital disease generally use anchors/nails, sutures, absorbable nails or gels for fixation. Due to low biological strength of the cartilage, the repaired cartilage or soft tissue is more likely to fall off, rehabilitation requirements are high, and the failure rate of the surgeries is high. The patient needs a second surgery, which has high cost, and multiple surgeries are not conducive to the rehabilitation of the patient. Moreover, in the traditional operation method, the suture operation takes a long time, the fixing effect is poor, and processing steps are cumbersome; the fixation strength of the absorbable nails is not matched with the strength of cartilage or regenerated cartilage; gel fixation strength is small; and anchor line fixation cannot effectively achieve good fixation of the cartilage due to the strength mismatch.

### SUMMARY OF PRESENT INVENTION

In view of the technical problems of inconvenient operation and poor fixing effect of a cartilage or soft tissue fixing method in the prior art, the purpose of the present invention is to provide a cartilage and soft tissue fixing device of network bridge-shaped structure having convenient operation and good fixing effect. The cartilage and soft tissue fixing device has the role of improving the fixing effect of cartilage, soft tissue, tissue engineering cartilage and cartilage repair materials.

The cartilage and soft tissue fixing device of the present invention comprises:

a plurality of anchors, wherein the anchors are composed of columns and vertebral apexes, the columns are evenly provided with bone growth holes (through holes) and/or bone growth slots (not communicated), the columns are further evenly provided with anti-falling limiting slots, and the columns are provided with line passing holes or line clamping slots near tail ends; and

network bridges, wherein the network bridges comprise peripheral bridges and inscribed bridges and the inscribed bridges are connected in the peripheral bridges.

The anchors are connected to the peripheral bridges by means of the line passing holes or the line clamping slots.

When the line passing holes are arranged on the tail ends of the anchor bodies, the line passing holes can be penetrated on the peripheral bridges in advance when manufactured before surgery, and can be directly nailed into cartilage or soft tissue during surgery. When the line clamping slots are arranged on the tail ends of the anchor bodies, the line clamping slots can be individually manufactured and packaged, and can be clamped to the peripheral bridges and fixed during surgery.

In addition, the shapes of the anchors can be adjusted according to actual needs.

In some preferred anchors of the present invention, the columns are cylinders, and the vertebral apexes are cones.

In some other preferred anchors of the present invention, the columns are prisms; the vertebral apexes are pyramids; and the edges of the prisms and the pyramids are chamfered.

Both the cylindrical anchors and the prismatic anchors can be provided with the line passing holes and can also be provided with the line clamping slots. One network bridge can only be configured with the anchors provided with the line passing holes or the anchors provided with the line clamping slots, and of course can be configured with the combination of two types of anchors. One network bridge can be configured with the anchors of the same shape or configured with the anchors of different shapes, that is, the anchors of various shapes can be used individually or in combination.

The shapes of the network bridges can be adjusted according to the contour area of the cartilage or soft tissue which is actually required to be fixed. In some preferred embodiments of the present invention, the peripheral bridges are circular, rectangular or special-shaped, wherein special shapes comprise polygons, ellipses, water drop shapes, eggplant shapes, and the like.

In some preferred network bridges of the present invention, the inscribed bridges are radiated, and comprise radial bridges and latitudinal bridges; and the distances between the latitudinal bridges are the same or are gradually widened from inside to outside.

In some other preferred network bridges of the present invention, the inscribed bridges are orthogonal network bridges or non-orthogonal network bridges, and other non-radiated network bridges.

In some preferred network bridges of the present invention, the network bridges are provided with anchor anti-slip structures and/or anchor anti-retrogradation structures. The anchor anti-slip structures may be selected from anchor installing hole connected with the peripheral bridges, or grooves provided on the peripheral bridges. The anchor anti-retrogradation structures can install concave-convex structures on one side or both sides of the anchor installing positions on the network bridges. Preferably, the concave-convex structures on one side or both sides can be designed as one-way inclined concave-convex smooth structures or toothed structures to ensure that the anchors can only slide in one direction and cannot retrograde.

The peripheral bridges are provided with anchor installing holes.

Further, the anchor installing holes are also connected with the inscribed bridges.

When the line clamping slots are arranged on the tail ends of the anchor bodies, the line clamping slots clamp the network bridges and opening directions face the direction away from the centers of the network bridges.

For materials, preferably, the network bridges are absorbable materials, and the anchors are absorbable materials or non-absorbable materials.

The absorbable materials may be selected from absorbable polymer materials, absorbable inorganic materials or absorbable composite materials.

The absorbable polymer materials comprise collagen, chitin, cellulose, polyesters (such as polylactic acid, polyglycolide-lactide copolymer, polycaprolactone, polydioxanone, and the like), polyamino acid and the like; and the absorbable inorganic materials comprise biodegradable ceramics such as tricalcium phosphate. The absorbable composite materials are materials formed by optimally combining more than two different absorbable materials, such as composition between polymer materials, composition between polymer materials and inorganic materials, and composition between materials and biologically active substances.

The present invention has the advantages as follows:

Firstly, the anti-falling limiting slots arranged on the columns of the anchors can effectively reduce the occurrence probability of backward slippage from the holes after the anchors are fixed, and improve the anti-falling effect of the anchors.

Secondly, the bone growth holes or bone growth slots arranged on the columns of the anchors can be used for the growth of cartilage or soft tissue in a rehabilitation process, and further improve the fixing effect of the anchors, thereby ensuring the fixing effect of the network bridges on the repaired cartilage or soft tissue.

Thirdly, the network bridges are composed of the peripheral bridges and the inscribed bridges; the inscribed bridges can effectively cover the cartilage or soft tissue region to be fixed, to prevent the cartilage or the soft tissue from falling; the peripheral bridges and the plurality of anchors can be used for effectively fixing the network bridges; and the fixed network bridges can provide the biomechanical strength of the cartilage repair period, better improve the fixing effect of the repaired cartilage or soft tissue, and reduce the probability of secondary surgery.

Fourthly, the anchors and the network bridges made of the absorbable materials can be absorbed by human bodies after rehabilitation without the need of removing, thereby avoiding the discomfort of patients caused by taking the anchors.

### DESCRIPTION OF THE DRAWINGS

FIG. 1 is a stereoscopic schematic diagram of a cartilage and soft tissue fixing device in embodiment 1 of the present invention;

FIG. 2 is a stereoscopic schematic diagram of I type anchor of the present invention;

FIG. 3 is a stereoscopic schematic diagram of II type anchor of the present invention;

FIG. 4 is a sectional schematic diagram of II type anchor of the present invention;

FIG. 5 is a stereoscopic schematic diagram of III type anchor of the present invention;

FIG. 6 is a side schematic diagram of III type anchor of the present invention;

FIG. 7 is a schematic diagram of concentric circular radiated equal-width network bridges of the present invention;

FIG. 8 is a schematic diagram of concentric circular radiated widening network bridges of the present invention;

FIG. 9 is a schematic diagram of concentric rectangular radiated equal-width network bridges of the present invention;

FIG. 10 is a schematic diagram of special-shaped radiated network bridges of the present invention;

FIG. 11 is a schematic diagram of concentric rectangular orthogonal network bridges of the present invention;

FIG. 12 is a schematic diagram of concentric circular non-orthogonal network bridges of the present invention;

FIG. 13 is a schematic diagram of eccentric circular non-orthogonal network bridges of the present invention;

FIG. 14 is an assembling schematic diagram of concentric rectangular orthogonal network bridges and anchors of the present invention;

FIG. 15 is an assembling schematic diagram of concentric circular non-orthogonal network bridges and anchors of the present invention; and

FIG. 16 is an assembling schematic diagram of eccentric circular non-orthogonal network bridges and anchors of the present invention.

### REFERENCE NUMERALS

I type anchor; line clamping slot 111; bone growth slot 112; anti-falling limiting slot 113;

II type anchor 12; line passing hole 121;

III type anchor 13;

concentric circular radiated equal-width network bridge 21; peripheral bridge 211; groove 212; radial bridge 213; latitudinal bridge 214; center 215;

concentric circular radiated widening network bridge 22; concentric rectangular radiated equal-width network bridge 23; special-shaped radiated network bridge 24; concentric rectangular orthogonal network bridge 25; concentric circular non-orthogonal network bridge 26; and eccentric circular non-orthogonal network bridge 27.

### DETAILED DESCRIPTION OF PREFERRED EMBODIMENTS

The present invention will be further described below in combination with specific embodiments. It shall be understood that the following embodiments are only used to illustrate the present invention, rather than to limit the scope of the present invention.

### Embodiment 1

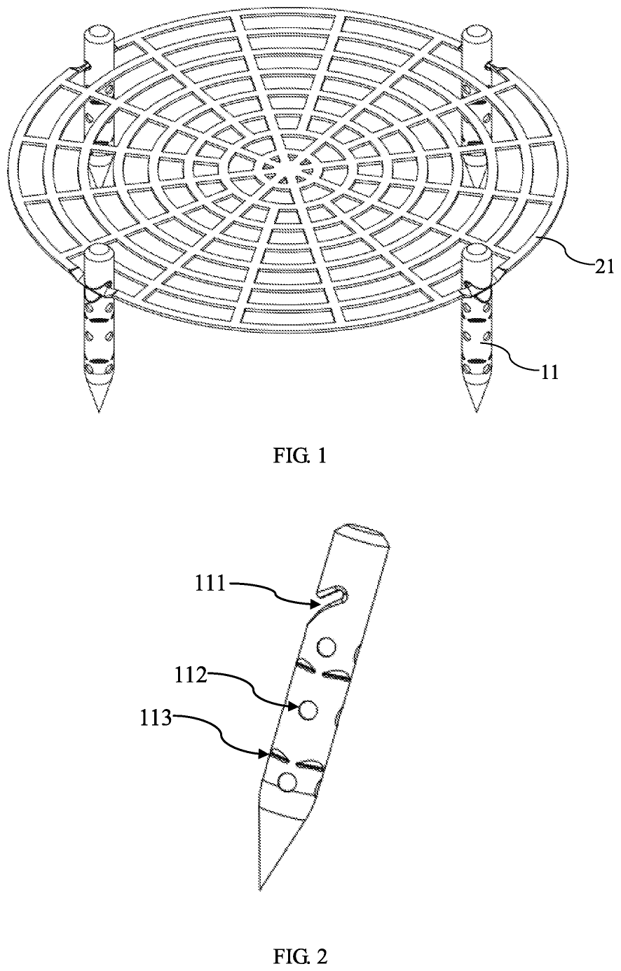

FIG. 1 shows a cartilage and soft tissue fixing device in a preferred embodiment of the present invention. The device comprises four I type anchors 11 and concentric circular radiated equal-width network bridges 21.

As shown in FIG. 2, the I type anchor 11 is composed of a column (cylinder) and a vertebral apex (cone); the column is provided with a line clamping slot 111 near a tail end; and the column (the middle section of the whole anchor) is evenly provided with bone growth slots 112 and anti-falling limiting slots 113.

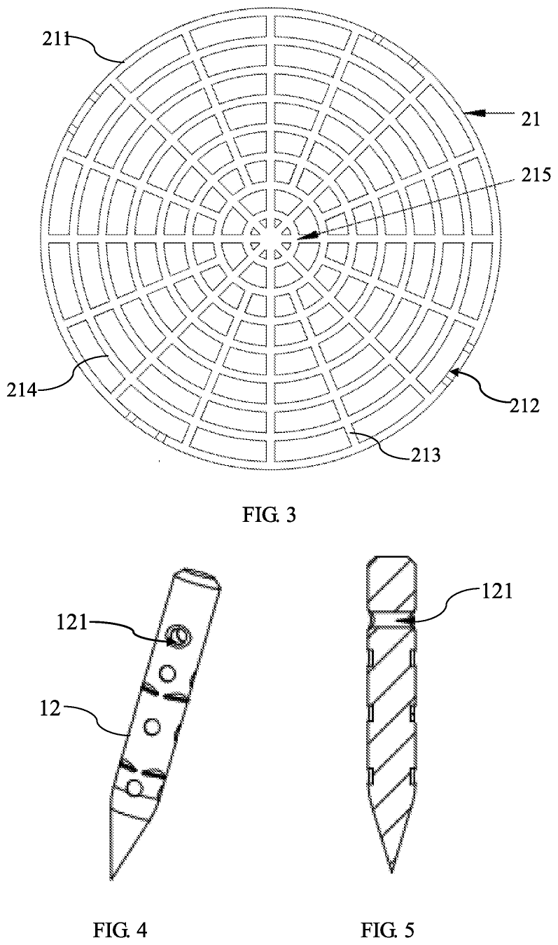

As shown in FIG. 3, a concentric circular radiated equal-width network bridge 21 comprises a peripheral bridge 211 and an inscribed bridge. The peripheral bridge 211 is provided with a groove 212 at the connection position for clamping the line clamping slot 111 of the I type anchor 11. The inscribed bridge is radially connected to the peripheral bridge 211 and comprises radial bridges 213 and latitudinal bridges 214; and the distances (radial widths) between the latitudinal bridges 214 are the same.

In actual use, the center 215 of the concentric circular radiated equal-width bridge 21 is used as a functional center, and the functional center is aligned with the main fixing region of the cartilage or soft tissue, to ensure that all cartilages or soft tissues to be fixed are within the coverage range of the concentric circular radiated equal-width bridge 21. Then, the I type anchor 11 is used to fix the periphery. It shall be noted that the opening direction of the line clamping slot 111 faces the direction away from the functional center.

In some other embodiments of the present invention, the I type anchor 11 can also be replaced with II type anchor 12 or III type anchor 13.

As shown in FIG. 3 and FIG. 4, the II type anchor 12 is different from the I type anchor 11 in that the line clamping slot 111 is replaced with a line passing hole 121. The rest is the same as the I type anchor 11. The II type anchor 12 needs to be connected to the network bridge in advance in manufacturing, so that the peripheral bridge passes through the line passing hole 121.

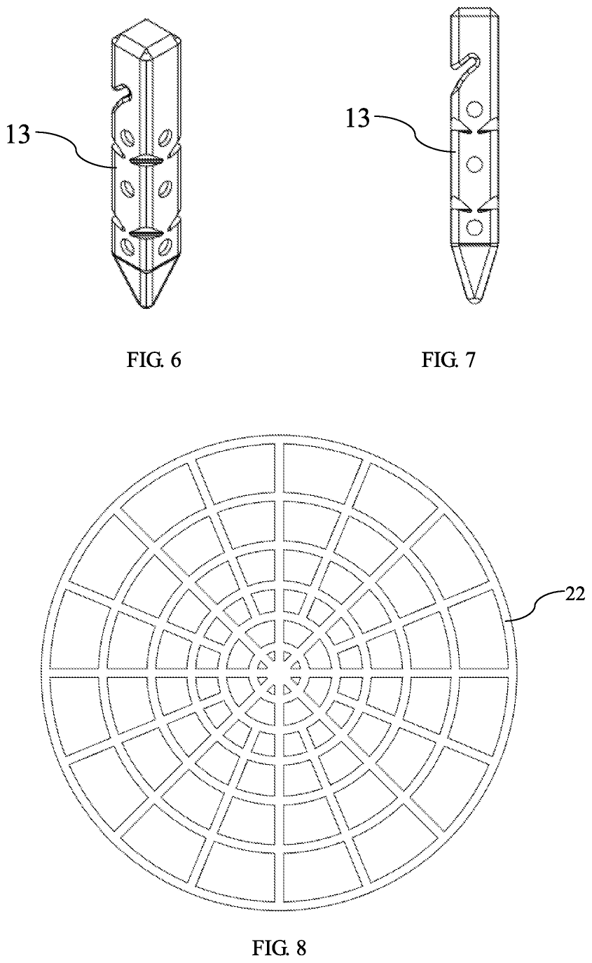

As shown in FIG. 5 and FIG. 6, the III type anchor 13 is different from the I type anchor 11 in that the columns are prisms; the vertebral apexes are pyramids; and the edges of the prisms and the pyramids are chamfered. The rest is the same as the I type anchor 11.

The above three anchors can be used individually or in any combination.

For example, the II type anchor 12 can be used in combination with the I type anchor 11 or the III type anchor 13, wherein the II type anchor 12 can be one or more, can be connected to the network bridge in advance before the surgery, and can be directly nailed into cartilage or soft tissue for fixation during the surgery, while the I type anchor 11 and the III type anchor 13 can be connected and fixed during the surgery. This combination makes the operation more convenient in the entire process of the initial positioning or the subsequent overall fixation.

In some other embodiments of the present invention, the network bridges covering the cartilage or soft tissue to be fixed can also be adjusted according to the shape and structure of the cartilage and soft tissue in the actual surgery, and the concentric circular radiated equal-width bridge 21 can be replaced with other shapes of network bridges. There are various shapes and types of network bridges which can be classified into concentric network bridges, eccentric network bridges and multi-center network bridges according to whether the centers of the network bridges are overlapped with the functional center (the position of the main fixing region of the cartilage or soft tissue is aligned in use); can be classified into circular network bridges, rectangular network bridges and special-shaped network bridges according to the structural types; and can be classified into radiated equal-width network bridges, radiated widening network bridges, orthogonal network bridges and non-orthogonal network bridges according to the radial width of the grids. FIG. 8 to FIG. 13 respectively show several preferred network bridges.

FIG. 8 shows concentric circular radiated widening network bridges 22, and the radial widths are gradually widened from inside to outside.

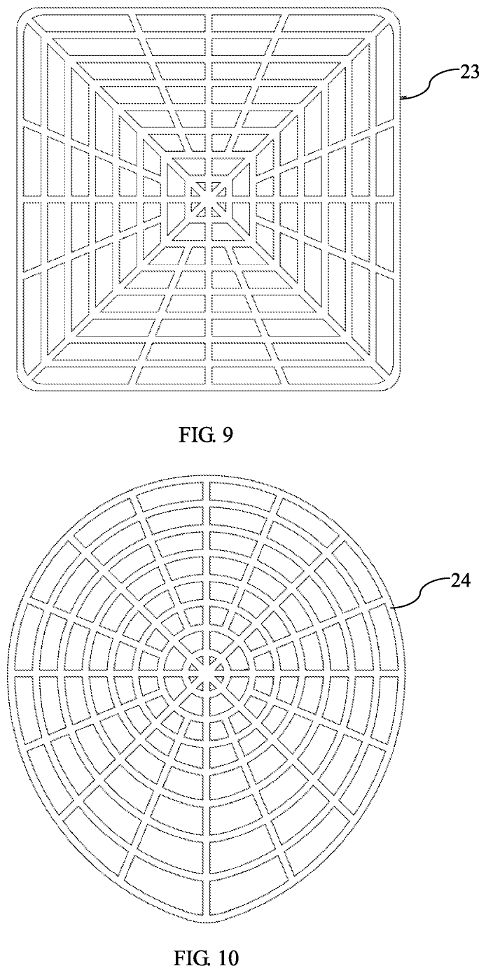

FIG. 9 shows concentric rectangular radiated equal-width network bridges 23.

FIG. 10 shows special-shaped radiated network bridges 24.

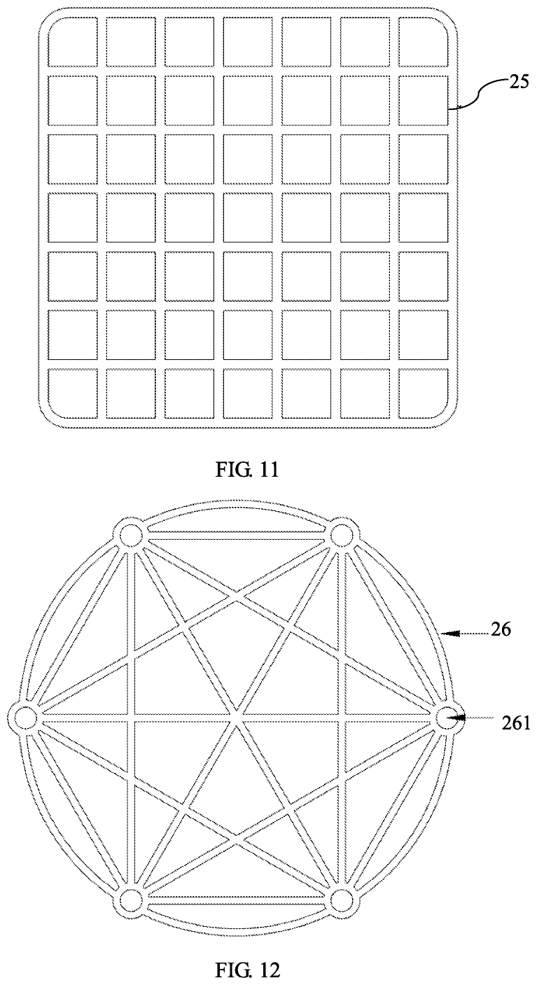

FIG. 11 shows concentric rectangular orthogonal network bridges 25, and the assembling of the network bridges 25 and the anchors is shown in FIG. 14.

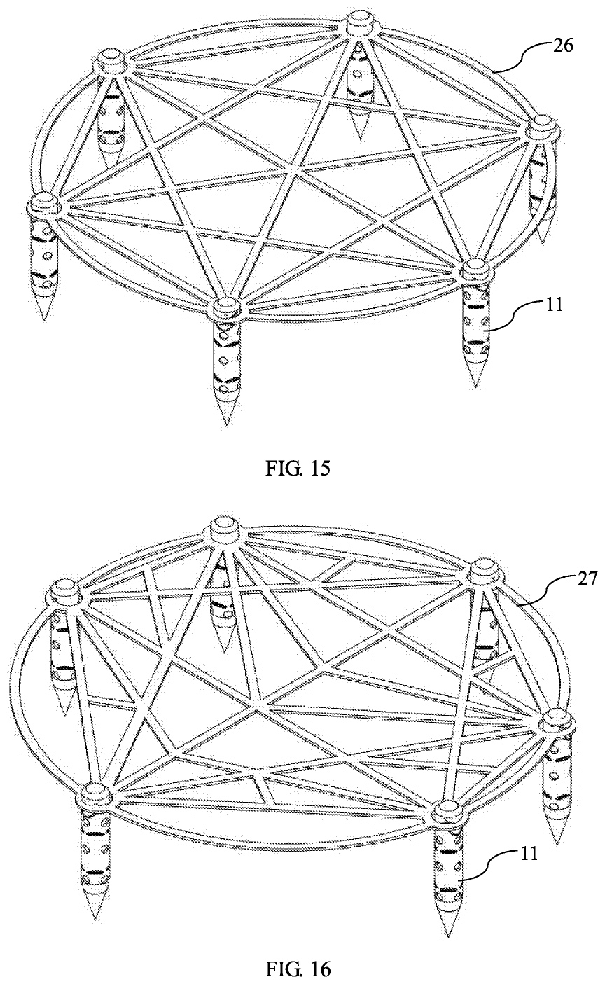

FIG. 12 shows concentric circular non-orthogonal network bridges 26; the peripheral bridges are also provided with anchor installing holes 261 at intervals; and the assembling of the network bridges 26 and the anchors is shown in FIG. 15. Of course, the anchors can be installed not only in the anchor installing holes 261, but also in other positions of the peripheral bridges.

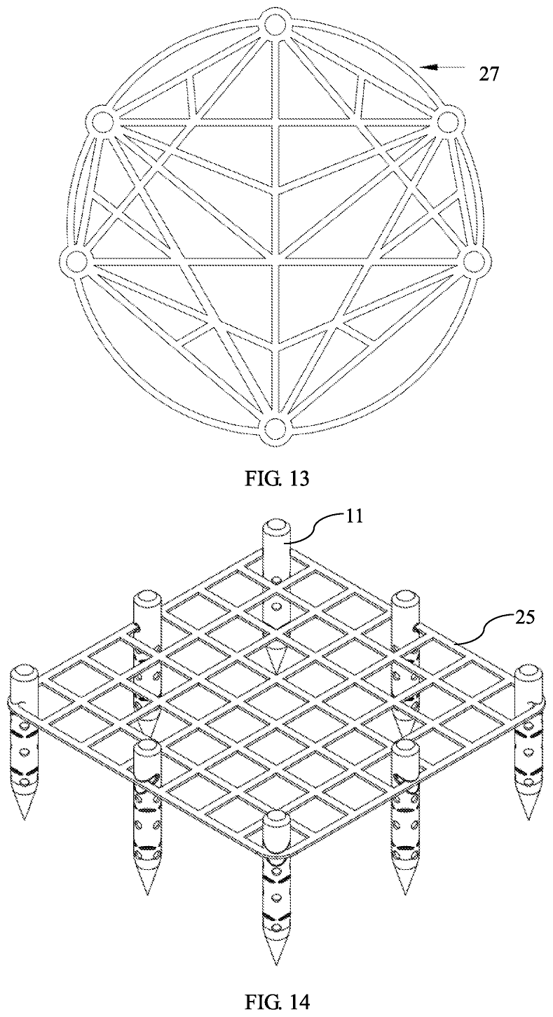

FIG. 13 shows eccentric circular non-orthogonal network bridges 27; the peripheral bridges are also provided with anchor installing holes at intervals; and the assembling of the network bridges 27 and the anchors is shown in FIG. 16. Of course, the anchors can be installed not only in the anchor installing holes, but also in other positions of the peripheral bridges.

The grooves or the anchor installing holes on the peripheral bridges are used as anchor anti-slip structures and can effectively prevent the anchors from slipping. In some other preferred embodiments of the present invention, the anti-retrogradation structures can also be arranged on the network bridges, and specifically can install concave-convex structures on one side or both sides of the anchor installing positions on the network bridges. Preferably, the concave-convex structures on one side or both sides can be designed as one-way inclined concave-convex smooth structures or toothed structures to ensure that the anchors can only slide in one direction and cannot retrograde.

The preferred embodiments of the present invention are specifically described above, but the present invention is not limited to the embodiments. Those skilled in the art can make various equivalent variations or replacements without departing from the innovation of the present invention. These equivalent variations or replacements are included in the scope defined by the claims of the present application.

We claim:

1. A cartilage and soft tissue fixing device, comprising:
a plurality of anchors, wherein the anchors are composed of columns and vertebral apexes, the columns are evenly provided with bone growth holes and/or bone growth slots, the columns are further evenly provided with anti-falling limiting slots, and the columns are provided with line passing holes or line clamping slots near tail ends; and
network bridges, wherein the network bridges comprise peripheral bridges and inscribed bridges and the inscribed bridges are connected in the peripheral bridges;
the anchors are connected to the peripheral bridges by means of the line passing holes or the line clamping slots.

2. The cartilage and soft tissue fixing device according to claim 1, wherein the columns are cylinders, and the vertebral apexes are cones.

3. The cartilage and soft tissue fixing device according to claim 1, wherein the columns are prisms; the vertebral apexes are pyramids; and the edges of the prisms and the pyramids are chamfered.

4. The cartilage and soft tissue fixing device according to claim 1, wherein the peripheral bridges are circular, rectangular or special-shaped.

5. The cartilage and soft tissue fixing device according to claim 1, wherein the inscribed bridges are radiated, and comprise radial bridges and latitudinal bridges; and the distances between the latitudinal bridges are the same or are gradually widened from inside to outside.

6. The cartilage and soft tissue fixing device according to claim 1, wherein the inscribed bridges are orthogonal network bridges or non-orthogonal network bridges.

7. The cartilage and soft tissue fixing device according to claim 1, wherein the network bridges are provided with anchor anti-slip structures and/or anchor anti-retrogradation structures.

8. The cartilage and soft tissue fixing device according to claim 1, wherein the line clamping slots clamp the network bridges and opening directions face the direction away from the centers of the network bridges.

9. The cartilage and soft tissue fixing device according to claim 1, wherein the network bridges are absorbable materials, and the anchors are absorbable materials or non-absorbable materials.

10. The cartilage and soft tissue fixing device according to claim 1, wherein the absorbable materials are absorbable polymer materials, absorbable inorganic materials or absorbable composite materials.
